(19) United States
(12) Patent Application Publication
(10) Pub. No.: US 2003/0091543 A1
(43) Pub. Date: May 15, 2003

(54) Therapeutic cell preparation grafts and methods of use thereof

(76) Inventors: Matthew B. Klein, Los Altos, CA (US); Charles B. Cuono, Chandler, AZ (US)

Correspondence Address: MINTZ LEVIN COHN FERRIS GLOVSKY & POPEO, 666 THIRD AVENUE, NEW YORK, NY, 10017, US

(21) Appl. No.: 10/044,004

(22) Filed: Jan. 11, 2002

(30) Foreign Application Priority Data
Oct. 26, 2001 (US) (24057-001 PRO)

(51) Int. Cl. A61K 48/00; C12N 5/08; C12N 15/85

(52) U.S. Cl. 424/93.21; 435/366; 435/455

(57) ABSTRACT

A biological preparation including genetically modified cells together with biocompatible matrices and methods of use thereof are provided. The biological preparation is useful in treating a subject at risk for or suffering from a disease in a controllable dosage and time-dependent manner, and for in vitro and in vivo screening of candidate drug therapies

### RELATED APPLICATION

This application claims priority to Express Mail No. EL831679505US, filed Oct. 26, 2001, which is incorporated herein by reference in its entirety.

### BACKGROUND OF THE INVENTION

1. Field of the Present Invention

The present invention generally relates to methods, preparations and pharmaceutical compositions for introducing and removing biological materials into and from mammalian subjects. More specifically, the present invention uses a living biological cell preparation graft to introduce therapeutic biological materials such as proteins into patients in need thereof.

2. Description of the Related Art

The easy accessibility of the skin makes this organ an exciting target for development of gene therapy protocols. Gene therapy involves the strategic manipulation of cells to produce increased amounts of endogenous proteins, or to produce exogenous or modified proteins or even bio-organic substances which are products of enzymatic biosynthesis, such as hormones. To date, gene therapy using keratinocytes or other constituent cells of the skin has focused upon treatment of cutaneous diseases and a variety of metabolic disorders, as well on the healing of superficial wounds. Thus far, full phenotypic reversion of a diseased phenotype has been achieved in vivo for junctional epidermolysis bullosa and X-linked or lamellar ichthyosis and in vitro for xeroderma pigmentosum. Further, phenotypic reversion of cells Isolated from patients suffering from a number of metabolic diseases such as gyrate atrophy, familial hypercholesterolemia or phenylketonuria is under study (See Spirito et al., (2001) J. Gene Med. 3:21-31). However, the sustained, controllable expression of transgenes in skin remains a primary limitation of the technology.

The skin can also be used as a bioreactor to synthesize and secrete proteins into the blood stream (See Cao et al., (2000) Hum. Gene Ther. 11:2297-300). Introduction into keratinocytes of recombinant nucleic acids encoding for secreted proteins holds promise as a method of treatment of systemic diseases. Still, difficulties with precise control of the amount and duration of the treatment remain to be overcome.

The use of locations other than the skin to introduce biological materials (e.g., recombinant proteins) derived from cells of the skin, elsewhere from the intended recipient, or from another mammalian source and introduced to the recipient in the form of an integrated graft is also of great significance. Introduction of biological material to regions such as the central nervous system via the dura matter or spinal meninges will allow circumvention of the blood-brain barrier, which currently provides a significant challenge to the treatment of neurological disorders. Likewise, delivery of therapies directly to the myocardium via the pericardial membrane would provide for precise targeting of cardiac treatments, and avoid undesirable systemic side effects. Targeted regional modular delivery of biologic therapies is also of great potential, as might be exemplified by the delivery of angiogenic substances to improve circulation in an ischemic extremity resulting from peripheral vascular disease, delivery of substances to fracture zones to accelerate bone healing, or delivery of therapies to the fetus via the amniotic membranes.

Accordingly, it is desirable to be able to provide to a mammalian subject a composition containing a genetically modified cell confined and integrated within a stable and durable structural matrix capable of secreting a therapeutic biological material such as a protein.

### SUMMARY OF THE INVENTION

The preparations and methods according to the present invention possess new capacities and abilities in the delivery of biological therapeutics to a mammal, such as a human subject in need thereof. The present invention enables the introduction of biological therapeutics such as recombinantly expressed proteins via a cell preparation, integrated with a structural, functional, biocompatible matrix in the form of a graft. The treatment can be either systemic, regional or focal, and the quantity and duration of treatment of these biological therapeutics can be dynamically modulated by regulating the number of functional gene copies per transformed cell, number of cells per unit volume of graft, and/or size of biologic preparation. Therapy dosage and duration can be stringently controlled and even promptly terminated by fractional or complete removal of the cell preparation graft, and/or incorporation of control elements in the nucleic acid cassette which may then be switched off/on via oral drug administration or by exposure of the cell preparation graft to physical or chemical stimuli such as UV light. Accordingly, the present invention provides a method of reversibly delivering an integral, defined and contained cell preparation graft to a mammal, whereby a cell preparation graft made up of one or more cells is introduced into a tissue of a mammal and then removed or rendered inactive after a defined duration of treatment.

The cell preparation grafts and methods according to the present invention are particularly suited for secreting recombinant proteins into the circulation or other region of a subject. The amount of recombinant protein introduced into the subject can be determined by the number of functional gene copies per modified cell, and/or the quantity of cells modified to express this protein which are present in the cell preparation graft, as well as other parameters which one skilled in the art would know to modify with a minimum of experimentation. Other biological therapeutics such as hormones, and other compounds such as drugs synthesized by the component cells of the cell preparation graft are within the scope of this invention. The cell preparation grafts of the present invention include modified cells which have been modified by the introduction of an endogenous or exogenous nucleic acid (e.g., DNA, RNA and ribozymes).

Accordingly, in one aspect of the present invention, a cell preparation graft includes one or more cells, which may be modified cells (e.g., keratinocytes expressing recombinant human Factor VIII) and a biocompatible matrix. The cells of the cell preparation graft may be taken from the subject (autologous) and grown in vitro prior to re-introduction in the subject, or the cells may be from other human sources (allogeneic), such as fetal tissue, or even from non-human sources (xenogeneic). When non-autologous cell sources are employed, appropriate supplementary modifications are made to these cells as may be necessary to render these cells immunologically acceptable to the intended recipient. In a related aspect, the cell preparation graft includes cells that are isolated from non-human mammalian sources, e.g., rodents, primates, cows and pigs, and non-mammalian sources.

Another aspect of the invention is a method of modulating the delivery of a compound to a mammalian subject by introducing into a tissue of the mammal and then removing, either fractionally or in entirety, a cell preparation graft containing one or more cells that have been modified such that they secrete a compound into the tissue. While in some preferred embodiments the mammal is a human, the present invention encompasses the introduction of cell preparation grafts into non-human mammals, including, for example, rodents, primates, cows and pigs, and other species of domestic livestock. In another aspect of the present invention, a method is disclosed for treating a subject suffering from or at risk of a disease. The method entails introducing an integral, defined cell preparation graft containing a biocompatible matrix and one or more modified cells that secrete a therapeutic compound into the tissue of said subject such that the disease is treated, then removing all or a portion of the cell preparation graft. The genetic modifications of the cells is accomplished through the incorporation of functional genetic information (genes) in the form of nucleic acids (e.g., DNA, RNA, ribozymes), generally derived from the same mammalian species as the intended recipient. However, nucleic acids from a broad range of allogeneic and xenogeneic origins may also be employed. In the case of non-autologous nucleic acids, the immunogenicity of the expressed proteins in the species of the intended recipient is determined. If necessary, appropriate modifications of the gene(s) are performed to reduce or eliminate the immunogenicity, while retaining therapeutic functionality of the expressed proteins.

Moreover, the above aspect may include further features and additional components of the cell preparation graft, including natural and/or synthetic biocompatible matrices, and markers (e.g., permanent pigments, fluorochromes, etc.) to identify the cell preparation graft. The cell preparation grafts may also contain functional control elements which are either constitutive or inducible via physical or chemical (e.g., drugs, UV light) stimuli to effect up or down regulation of therapy, which can be administered via the gastrointestinal tract (e.g., orally, suppository), parenterally (e.g., intramuscular, intravenous, subcutaneous) or topically. Another aspect of the invention is a method of identifying a candidate agent that modulates the measurable effect of a compound that is delivered to a mammalian subject by introducing into a tissue of the mammal a cell preparation graft containing one or more cells that have been modified such that they secrete a compound into the tissue. A measurable effect is any physical, chemical, biochemical or biological change that can be measured or detected in a cell or tissue of a mammal. In alternative embodiments, this method is performed in vivo, ex vivo or in vitro. The present invention also pertains to the candidate agents identified by this method.

Also within the scope of the invention are compositions which include a cell preparation graft made up of one or more cells. In certain embodiments the cell preparation graft includes genetically modified cells, preferably cells that have been modified to secrete recombinant proteins, peptides, or even non-protein bio-organic substances, and a biocompatible matrix. In other embodiments the cell preparation graft includes other compounds such as drugs (e.g., anti-inflammatories, antibiotics, anti-virals, anti-neoplastics and anti-mycotics).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

### DETAILED DESCRIPTION OF THE PREFERRED EMBODIMENTS

The object of this invention is in part to provide a novel method of gene therapy, employing ex vivo genetic engineering of mammalian cells, preferably human, to establish within those cells one or more genes or other nucleic acid followed by the introduction of these modified cells into a mammalian host, preferably human, as a prefabricated, integral lamellar unit (“cell preparation graft”). The dosage of the therapeutic agent which is the product of the incorporated nucleic acid or is a resulting product of the action of the incorporated nucleic acid is controlled by modification of parameters, including, but not limited to: graft size, population density of transformed cells resident within the graft, and the rate of production of the therapeutic agent by the transformed cells. The rate of production of the therapeutic agent by the transformed cells is, in turn, controllable by appropriate design strategy, which is the selection of nucleic construct, choice of transformation method, arrangement of the DNA or RNA cassette and its introduction into the host cells, as will be described herein. Briefly, this would include, but is not limited to: appropriate selection of vectors, cassette design, promoters, enhancers and polyadenylation sequences. An important and novel feature of this invention is the ability to also control duration of therapy via facile surgical removal of all or a portion of the graft, and/or modifications in design strategy which can provide for inducible promotion or cessation of transcription, translation and secretion of the therapeutic agent of interest.

1. Methods of Treatment.

In a First aspect, the invention is a method of introducing a cell preparation graft comprising one or more cells to a mammal suffering from or at risk of a disease. This method is useful to isolate cells from a mammal, culture the cells in vitro, genetically modify the cells, and then integrate the cells on the surface of, or within, a biocompatible matrix of variable structure and composition prior to introduction of the modified cells into a mammal as a discrete, size-controllable, and removable graft. The source of the cells for modification may be a human or non-human mammal or even a non-mammalian source. Any human or non-human mammal may be considered as a candidate recipient of a cell preparation graft.

1.1 Source of cells. Any mammalian or non-mammalian cell type which is capable of being maintained under cell culture conditions, and preferably expansion in number, in vitro, and subsequent integration into a graft form (to be described) would be included in this invention. In preferred embodiments, the cells are isolated from and re-introduced into the same animal (autologous cells, i.e., cells obtained from the intended recipient), thus avoiding immune rejection, and disease transmission. In other embodiments, the cells are isolated from allogeneic embryonic or neonatal tissue, which is inherently less immunogenic than adult tissue. In other embodiments this invention also includes the use of so-called immunologically neutral allogeneic cells, i.e., allogeneic cells of either fetal or adult origin which themselves have been genetically modified to eliminate the synthesis and/or expression of the cell surface antigens which are responsible for the self/non-self recognition by the immune system of the recipient. These antigens are chiefly within, but not limited to, the major histocompatibility complex (MHC), Classes I and II. In some embodiments of the first aspect, cells isolated as part of the method can be from humans or other mammals (e.g., rodent, primate, cow or pig) or non-mammalian sources. In other embodiments, these cells can be derived from skin or other organs, e.g., heart, brain or spinal cord, liver, lung, kidney, pancreas, bladder, bone marrow, spleen, intestine, or stomach. In other embodiments, these cells can be stem cells which, in culture, can be differentiated into a desired cell type. The surgical introduction and removal of cells from a particular tissue or organ of interest is well known to those skilled in the art and is facilitated by burgeoning field of endoscopic surgical techniques, which now provide access to these sites with minimal invasiveness.

1.2 Type of cells. In some embodiments, the isolated cells are, e.g., epidermal keratinocytes, oral and gastrointestinal mucosal epithelia, urinary tract epithelia, as well as epithelia derived from other organ systems, skeletal joint synovium, periosteum, bone, perichondrium, cartilage, fibroblasts, muscle cells (e.g. skeletal, smooth, cardiac), endothelial cells, pericardium, dura, meninges, keratinocyte precursor cells, keratinocyte stem cells (e.g., NIKS™), endothelial cells, pericytes, glial cells, neural cells, amniotic and placental membranes, stem cells, and serosal cells (e.g., serosal cells lining body cavities).

1.3 Source of Cell Modifying Agents. The nucleic acid to be incorporated into the target cells would preferably be obtained from the same mammalian species as the intended recipient. This is not, however, an absolute restriction in this invention. In the case of nucleic acids of xenogeneic origin, one skilled in the art could, with limited testing, determine the immunogenicity of the translated proteins in the species of the intended recipient. Similarly, one skilled in the art could determine the immunogenic epitopes of such xenogeneic proteins, and modify the nucleic acid cassette accordingly to reduce or eliminate (in the case xenogeneic proteins to be inserted into a human patient, the proteins are “humanized” using techniques well known in the art) the immunogenic epitope(s) and yet retain therapeutic functionality of the translation product.

The DNA to be incorporated can also be selected to direct the synthesis of directly or indirectly therapeutic products of transcription or reverse transcription. In a preferred embodiment, the incorporated DNA construct would direct the transcription of an anti-sense RNA which would be homologous with and bind to a specific mRNA, thereby blocking its translation into protein. In another preferred embodiment, the incorporated DNA would direct transcription of ribozyrnes, i.e., molecules of RNA which exhibit specific enzymatic action directed at cleavage of mRNA, thereby blocking protein translation. Alternatively, if a retroviral vector is employed, one could introduce RNA into the cells which could then direct the reverse transcription of a DNA which could bind or sequester to a synthetic or regulatory receptor-protein, thereby effecting a therapeutic action.

1.4 Modification of cells. In some embodiments of the first aspect, cells isolated as part of the method are genetically modified such that they express an exogenous protein, e.g., a full-length protein, a fusion protein, a mutated protein, a truncated protein, or a modified protein such as a phosphorylated protein; or to induce the expression of an endogenous protein. In other embodiments, cells can be modified to express antibodies and protein complexes. In yet other embodiments, cells can be modified to produce a therapeutic compound, such as a drug, or a detectable signal, such as green fluorescent protein (GFP), or a combination thereof. Cells can also be modified to express enzymes or any other protein that are involved in synthesis of therapeutic non-protein, bio-organic substances (“natural products”), such as therapeutic steroids (e.g., estrogens, androgens, mineralocorticoids, glucocorticoids, and anabolic steroids), and other therapeutic natural products. Cells can be modified to secrete a natural product, which is defined herein as any biosynthesized organic compound that is not a high molecular weight carbohydrate, a complex lipid, or a protein. Likewise, therapeutic non-mammalian natural products whose synthesis and secretion are directed by enzymes and proteins, such as tamoxifen, vincristine and digitalis glycosides, can be produced by mammalian cells carrying the appropriate exogenous genetic repertoire. Cells can also be modified to express a “suicide protein”, such as herpes simplex virus type 1 thymidine kinase (See e.g., Deglon, Human Gene Therapy 1996;7:2135) which would be sensitive to the antiviral agent gancyclovir. Cells can be modified to express more than one protein and/or compound concurrently or consecutively. The modification of cells can occur by any method known to one skilled in the art, such as e.g., transfection, transduction, microinjection, and other physical and chemical means well known in the art.

A genetic modification includes the introduction of an exogenous nucleic acid such as DNA, or the introduction of increased amounts of a normally endogenous nucleic acid. The latter situation would occur, for example, when the goal is to augment production of the therapeutic gene product, or to correct for a genetic dysfunction/non-function of the recipient's native gene. In the case of DNA, the DNA to be incorporated into the target cells can be any of a broad range of constructs, selected to achieve expression of either therapeutic products of translation, or products of transcription. In some cases, the introduced DNA can be considered endogenous, i.e., it is normally expressed in a cell but by reason of disease, disorder or other events, it is defective (e.g., mutated), produced in insufficient quantity, or not at all. In such cases, the DNA effecting the modification is correcting a genetic deficit. In a preferred embodiment, the construct would include an entire gene encoding for a functional translation product, e.g., functional, regulatory, or structural protein, peptide, cytokine, antibody, cell surface receptor, enzyme.

In another embodiment, the construct would include a plurality of heterologous genes (i.e. genes not expressed in a cell under physiological circumstances), acting in concert to produce the array of enzymes and regulatory proteins, which direct the synthesis and secretion of a therapeutic non-protein bio-organic substance. Examples of this strategy would include, but not be limited to, delivery of long term cancer therapy and chronic disease therapy (e.g., arthritis, cardiac disease) with molecules such as tamoxifen, vincristine, digitalis glycosides and warfarin (See e.g., Uchida, Dev Neurosci 1992;14:173, During, Gene Therapy 1998;5:820). In another embodiment, the plurality of genes may be functionally independent but inter-related as the needs of the recipient dictate. By example, a construct could be designed by anyone skilled in the art, to include the genes for both the low density lipoprotein (LDL) receptor and antithrombin for use in a patient suffering from or at risk of developing hypercholesterolemia and also at risk for vascular thrombosis.

In another preferred embodiment, a cell preparation graft would include a plurality of modified cell types, each producing a different therapeutic substance at a desired therapeutic level. This embodiment is useful as a screen for drug development. By way of non-limiting example, a number of different subsets of human fibroblasts and/or synovial cells can be altered to produce a spectrum of inflammatory substances. These subsets are then integrated into a graft matrix and used in vitro or in vivo in animal models to test/screen candidate anti-inflammatory drugs. This is accomplished through maintaining the cell preparation graft under cell culture conditions, exposing it to the candidate anti-inflammatory drug, and by monitoring changes in the production of the inflammatory substances via standard assays. For in vivo applications, the cell preparation graft is introduced into a suitable experimental animal, in which both clinical and chemical parameters can be monitored in response to treatment with the candidate drug(s). The integrated cell preparation graft, using human cells, offers obvious advantages over conventional drug screen assays.

In some especially preferred embodiments, cells of a single cell type may be segregated into subsets, with each subset undergoing a different genetic modification, and the subsets then integrated into a single cell graft preparation to deliver one or more therapeutics. Genetic modifications include introduction of one or more nucleic acid vectors, each vector causing the expression of one or more recombinant proteins. Such a cell preparation graft will confer both active and immediate passive immunity to a recipient when the incorporated nucleic acids direct the synthesis and expression of gene products which are immunogens (e.g., protein antigens, immunoglobulins, immunogens for anthrax, ebola virus, prions, and bubonic plague), and gene products which are specific neutralizing antibodies (See e.g., U.S. Pat. No. 6,214,804). The cells to be modified can also be made immunologically neutral prior to genetic modification, thereby creating a spectrum of “vaccine cell lines” which can be introduced in cell preparation graft form, and used as part of a broad-spectrum, polyvalent vaccine. A compound or plurality of compounds act as a vaccine if they stimulate the production of antibodies.

The genetic material to be introduced into the target cell most typically embodies a DNA sequence encoding a protein translation product. One skilled in the art could, without significant experimentation, determine the additional necessary elements such as promoters (universal or tissue specific, strong or weak, constitutive or inducible), termination signals, and regulatory sequences. Additionally, one skilled in the art could determine selection and placement of appropriate enhancers within the nucleic acid construct or cassette in order to regulate the basal transcription level governed by the chosen promoter. Any of a spectrum of polyadenylation sequences known to the art could be selected and engineered into the construct. Likewise, the construct could include specific introns, also well known in the art, which could upregulate expression of the nucleic acid of interest. In a preferred embodiment, the construct would include a so-called “marker gene” which would assist in the ex vivo selection of the cells containing or expressing the nucleic acid construct. Again, one skilled in the art could determine the appropriate marker, depending upon the nucleic acid construct selected and the target cell of interest. Typically, markers selecting for antibiotic resistance, e.g., ampicillin, neomycin, as well as the β-galactosidase and luciferase genes are well known in the art.

Once the architecture of the nucleic acid construct has been established, the next step is the selection of the delivery method to the target cells. A myriad of vectors well known in the art, can be used in accordance with this invention, and some are available commercially. Choice of method is dictated by a number of factors, including, but not limited to the size of the nucleic acid cassette, the replicative potential of the target cell, the duration of the proposed therapy, and the site of introduction of the cell preparation graft. The mammalian target cell can be transformed through a viral mechanism (transfection), using replication-deficient viruses, or through physical-chemical means (transduction) (See e.g., Sambrook, Molecular Cloning: A Laboratory Manual, 3^{rd} Edition). In the realm of transfection, preferred embodiments would include the employment of either DNA viruses or RNA viruses (retroviruses). The chief advantages of retroviral vectors as known in the art is the stable integration of the construct into the host cell genome. This provides for more efficient and long duration expression of the translation product(s), and for inheritance of the exogenous nucleic acid when the target cell divides. The well-known disadvantages of retroviral vectors include their limitation to use in dividing cells, and the potential for insertional mutagenesis, i.e., the random manner of the insertion of the nucleic acid construct may result in the disruption of a normal and desired cell growth regulatory site, thereby triggering uncontrolled cell growth. This potential disadvantage of retroviral vectors is, however, substantially of less concern in this invention by reason of the fact that the modified cells are collectively administered as an integral unit, a cell preparation graft. As such, the graft can be removed partially or in its entirety should any adverse consequence develop in any one or more of the modified cells. Other methods of treatment with modified cells have relied on administration methods, such as injection, which do not offer this stringency of control of the administered cell population. As well known to the art, many replication-deficient mammalian DNA viruses have been engineered to facilitate transfection of desired nucleic acid constructs. The adenovirus, and the adeno-associated viruses, in particular are preferred embodiments, since they are capable of efficient transformation of slowly dividing, and even terminally differentiated cells. Integrated forms of the adeno-associated virus carry the same benefit of stable integration of the DNA into the target cell genome as do retro-viral vectors, with the added advantage of utility in slowly dividing cells. Additionally, non-mammalian DNA viruses have been recruited to the task of infecting mammalian cells for the purpose of effecting their expression of exogenous genes (See U.S. Pat. No. 6,281,009).

Under some circumstances, it may be desirable to introduce the nucleic acid construct into the target cell without having it become integrated into the target cell genome. Rather, the construct remains as an independent, functional unit, referred to as an episome. Depending upon the therapeutic goal, e.g., long term or short term therapy, choice of therapeutic agent, episomal expression may be a desirable goal. Exemplary situations would include those target cells whose expression of the desired therapeutic agent is enhanced by episomal gene locus. Additionally, cell types which are unable to integrate the construct or which prove unstable, may be ideal candidates for the episomal introduction. The manner of construction of episomally introducible nucleic acid constructs is well known in the art. It is also within the scope of this invention to have a cell preparation graft consisting of cells containing both episomal and non-episomal nucleic acids.

Under some circumstances, it may be desirable to introduce a DNA construct into the genomic DNA of a target cell at a defined, pre-determined site, through homologous recombination (See U.S. Pat. No. 6,187,305 B1). This strategy may be of utility to activate genes which are constitutive but not expressed in the target cell, or which are expressed at sub-therapeutic levels. Targeted replacement or modification of the native regulatory sequence of the non- or hypo-functional gene with an activating regulatory sequence (e.g., from another gene in the same or another species, or an entirely synthetic regulatory sequence) results in the desired up-regulation (expression) of the native gene.

As noted above, the nucleic acid constructs may be also be introduced into the target cells through non-viral or transductive mechanisms. Exemplary techniques include electroporation, liposome transduction, and microinjection, all of which are well known to the art. The nucleic acid may also be introduced via chemical adducts, coupling the DNA or RNA to a carrier molecule (e.g., dextran, poly-lysine, DEAC-dextran, lipids, proteins, antibodies) which then facilitates trans-membrane delivery to the target cell. Other, well established methods may also be employed to effect transformation of the target cells with the nucleic acid construct of interest. These would include, but not be limited to calcium phosphate precipitation, and polybrene precipitation. Some of these agents are commercially available, e.g., Lipofectin™ (Bethesda Research Labs, Bethesda, Md.). The array of techniques to effect transformation of the target cell with the nucleic acid construct of interest is well known in the art, and the specific techniques themselves form no part of this invention. Further, it is understood that the efficiency of the various transformation methodologies is a function of a number of parameters, including the target cell type. Selection and optimization of transformation technique, however, is well known to the art, and is within the mean scope of one in the art.

Exemplary gene products and diseases amenable to treatment using this invention are indicated in Table 1.

TABLE 1

| DISEASE OR | |
|---|---|
| PHYSIOLOGIC STATE | THERAPIES |
| Diabetes, altered | Insulin, insulotropin, glucagon |
| glycemic states | |
| skeletal growth | human growth hormone |
| retardation | |
| Anemia | Erythropoietin (EPO), hemoglobins |
| Obesity | Ob gene translation product (leptin) |
| Immunodeficiency | adenosine deaminase, purine nucleoside |
| (e.g., AIDS) | phosphorylase, CD-4 |
| Hemophilia A | Factor VIII |
| Hemophilia B | Factor IX |
| Emphysema | α₁-antitrypsin |
| Hypercholesterolemia | LDL receptor protein |
| pernicious anemia | Intrinsic factor |
| glycogen storage | Glucose-6-phosphatase |
| disease | |
| Hypoalbuminemia | Albumin |
| Gaucher's disease | B-glucosidase (glucocerebrosidase) |
| cystic fibrosis | CF transmembrane conductance regulator |
| cardiovascular disease | Tissue Plasminogen Activator (tPA), |
| | urokinase, streptokinase, antithrombin III, |
| | Apolipoproteins (e.g., APO B48, A1), Low |
| | Density lipoprotein receptor, vascular |
| | endothelial growth factor (VEGF) |
| Calcium mineral diseases | Calcitonin, parathyroid hormone (PTH), PTH- |
| | like hormone |
| Severe Combined | adenosine deaminase |
| Immunodeficiency | |
| (SCID) | |
| Phenylketonuria | phenylalanine hydroxylase |
| von Willebrand's disease | von Willebrand Factor |
| cancers, cancer | Tumor Necrosis Factors (TNFs), cytokines, |
| suppression | anti-neoplastic agents (e.g., vincristine, |
| | doxorubicin, tamoxifen, methotrexate), |
| | interleukins (ILs), interferons (INFs), p53 and |
| | related, anti-BRCAs, anti-VEGF, anti- |
| | Epidermal Growth Factor (EGF), oncogene |
| | anti-sense RNAs |
| Peripheral vascular | VEGF, endothelins |
| disease | |
| neurodegenerative states, | Ciliary Neurotrophic Factor (CNTF), Brain |
| and post neural | Derived Neurite Factor (BDNF), Neurite |
| trauma conditions | Growth Factor (NGF), tyrosine hydroxylase. |
| retarded fracture healing | bone morphogenic proteins (BMP) |
| lactose insufficiency | Lactase |
| wound healing | Epidermal Growth Factors, Transforming |
| | Growth Factors, Granulocyte-Colony |
| | Stimulating Factors, Fibroblast Growth |
| | Factors, Interferons, Interleukins, Insulin-like |
| | growth Factors |
| thrombosis, | antithrombins, urokinases, tPAs , hirudins, |
| hypercoagulability | streptokinase |
| diabetes insipidus | Antidiuretic hormone (ADH) |
| Psychiatric Disorders | Selective Serotonin Reuptake Inhibitors, anti- |
| | psychotic bio-substances |
| Pain Control | Endorphins |
| Endocrineopathies | Estrogens, Androgens, mineralocorticoids, |
| | glucocorticoids, anabolic steroids, etc. |
| Hypothyroidism | Thyroid hormones, thyroglobulins |
| Muscular dystrophy | dystrophin |
| Naïve immune state to | specific antigens to confer active immunity, |
| pathogens | specific immunoglobulins to confer passive |
| (e.g., pathogenic | immunity |
| bacteria, fungi, viruses, | |
| and prions) | |
| Infections | anti-microbial polypeptides |
| (bacterial, fungal, viral) | |
| Shock, Sepsis | Lipid Binding Protein (LBP) |
| Leukemia | L-asparaginase |
| Disorders of digestive, | Pepsin, trypsin, chymotrypsin, |
| pancreatic states | cholecystokinin, sucrase, |
| | carboxypeptidase |
| Autoimmune states | Immunogens to induce tolerance, blocking |
| | antibodies |
| Oxidative Stress, | Catalase |
| Neurodegenerative | |
| Disorders | |
| Hypouricasemia, Gout | Uricase |
| Ehlers Danlos | Elastase |
| Thrombocytopenia | Thrombopoietin (TPO) |
| SCID/ADA deficiency | adenosine deamidase |
| porphyria | porphobilinogen deaminase |
| inborn errors of | specific enzymes catalyzing transformations at |
| carboxylic and amino | genetic block points, (e.g., glutaryl CoA |
| acid metabolism, | dehydrogenase) |
| (e.g., glutaric acidemia) | |
| homocystinuria | cystathionine B-synthase |
| Wilson's Disease, | specific copper transporting ATPase's |
| Menke's Disease | |
| Thalassemia | β-globin |
| Sickle Cell Anemia | α-globin |
| Baldness | Sonic hedgehog gene products |

1.5 Introduction of modified cells. The ability to introduce into a suitable mammalian recipient in modular form (“graft”), and to subsequently remove all or a fraction of the therapy-delivering transformed cells is an important, novel part of this invention. Depending upon the cell type and the locus of graft application, the constructs can take a number of forms. In a preferred embodiment of the first aspect of the invention, the cell preparation graft of modified cells and biocompatible matrix is introduced into the skin of the mammal. In other embodiments, the modified cells are introduced into e.g., the dura mater, the pulmonary cavity lining, the pericardial sac, the esophageal lining, the gastrointestinal lining, the peritoneal lining, urinary tract lining, the amniotic and/or placental membranes, the synovial joint space, periosteum, bone, muscle, fascia or the vasculature. More than one cell type can be introduced concurrently or consecutively, and more than one method of introduction can be employed. In still other embodiments, the cells are in the form of a sheet comprising one or more layers of cells or a graft comprising one or more layers of cells. The biocompatible matrix can be composed of natural or synthetic, and organic or inorganic material. By way of non-limiting example, the cell preparation graft to be introduced includes one or more cells and proteins such as extracellular matrix proteins, cell surface receptors, extracellular matrix-degrading enzymes, growth factors and cytokines, and enzymes involved in steroid synthesis; biocompatible polymers such as natural and synthetic biopolymers; and adhesive materials, such as peptides, glycopeptides, and natural and synthetic adhesives. A cell preparation graft may contain more than one cell type, each cell type being separately modified such that a different compound is produced. Each cell type may have more than one modification, e.g., may have episomal nucleic acids and non-episomal nucleic acids. In turn, each nucleic acid may encode for one recombinant protein or a plurality of recombinant proteins. While in some preferred embodiments the cell preparation graft is introduced into a mammal, the graft is also useful in vitro and ex vivo, e.g. for preclinical/laboratory screening and/or drug development and testing.

In one particularly preferred embodiment, a quantity of the above-described transformed human keratinocytes, after culture expansion to the desired total cell number and functional gene density, are applied to a suitable dermal matrix in vitro. The composite construct is then carried in cell culture for a short period of time to effect integration of the keratinocyte layer with the dermal matrix. The duplex construct is then ready for application as a graft to the intended recipient. Ideally, the keratinocyte source is autologous, i.e., the donor is the eventual intended recipient. The surgical procedures for harvest of the keratinocyte can be conducted under brief local anesthesia, and it can be conducted in a concealed body locus to obviate any visible scarring. Likewise, the application of the graft containing the transformed therapeutic keratinocytes, can also be conducted under local anesthesia. The graft can be applied to a concealed body region which is still accessible, should the need arise to remove a portion of the graft to thereby tailor the dosage, and/or to remove all of the graft when treatment is complete or for any other reason.

In this embodiment, any of a number of dermal matrices can be employed. One of these could be freshly prepared deconstructed allogeneic human dermis, devoid of any residual epidermal elements, and purged of any residual dermal cells. This material has been demonstrated to be a suitable recipient bed for cultured human epidermal cells, allowing restoration of normal epidermal-dermal junctional integration (See Proc 22^{nd} Am Burn Assn, 1990:6, Proc 23^{rd} Am Burn Assn, 1991:1, J Invest Dermatol 1991;97:843). Alternatively, a commercially available devitalized allogeneic human dermis can be employed in a similar manner (Alloderm™ LifeCell). Since the constructs of transformed keratinocytes plus human dermis allow for the most natural restoration of dermal-epidermal integrity, and hence the most normalized dermal-epidermal interactions, this would be a preferred embodiment for long-term gene therapy.

For more transient gene therapy a graftable construct consisting of the cultured transformed keratinocytes applied to commercially available collagen-glycosaminoglycan matrices could be considered (e.g., Integra T, Integra Life Science, Plainsboro, N.J.). In this construct, the keratinocytes would be more easily removable at the conclusion of therapy, as there would be no discrete dermal base harboring grafted transformed keratinocytes. Removal could be achieved through light topical anesthesia and superficial dermabrasion.

The grafting of transformed cells to other sites could be accomplished in a manner similar to that described for keratinocytes. Specifically, the transformed cells would be plated or seeded onto a dermal template or facsimile, allowed to attach and integrate and then could be grafted to the desired location. This approach could be applied to a broad and diverse range of body regions and organ systems, exemplified by the oral cavity, dura mater, pericardium, pleural surfaces, peritoneum, and urinary bladder. In each of these areas preferred embodiments would be either the selective abrasion of the intended graft recipient site in order to prepare a suitable vascular bed onto which the fabricated graft is applied and secured, or fenestration of the recipient area tissue (“windowing”), and application of the graft within the windowed zone. The graft may be secured either with sutures or with commercially available tissue adhesives. In more restricted areas, such as articular surfaces of joints, synovial membranes, etc., the transformed cells could be applied to a structural protein matrix whose composition mimics the intended environment. Techniques for preparation of matrices are well known to the art. (See, e.g., Proc Natl Acad Sci 1979;76:11274; Surgery 1994,115:633; J Bum Care Rehabil 1994;14:485). The transformed cell sheets, integrated with a structural matrix could then be grafted onto the prepared recipient bed. In joints, this could be accomplished via arthroscopic abrasion of the articular surface to prepare a suitable graft bed, followed by graft application and fixation, preferably with a biodegradable tissue adhesive. In other sites, where possible, a preferred embodiment would be the use of minimally invasive endoscopic techniques, both for grafting, and for graft removal if required for termination of treatment.

The invention does not require nor mandate the use of the same cell type as the native cell in the intended recipient area. A construct of transformed oral mucosal cells or gastrointestinal mucosal cells combined with a suitable matrix could in one embodiment, for example, be grafted into a site in the pleural cavity, using thorascopic techniques. Selection of the cell type is dictated primarily by the therapeutic goal. For example, if the therapeutic goal is short term local cytokine secretion into a cavitary site, an appropriately transformed intestinal epithelial graft may be better able to deliver the cytokine locally via its native apical secretory mechanisms, as opposed to grafting a serosal cell preparation graft which would predominantly if not exclusively deliver the synthesized protein systemically via the graft bed vascular connections. Those skilled in the art will recognize, or will be able to determine with standard experimentation, the appropriate target cell for selection, transformation and use as a removable graft at a specific locus.

In a preferred embodiment of this invention, the biocompatible matrix into which the transformed cells are integrated would contain pharmaceutical grade non-toxic, inert tattoo pigments embedded around its borders, as well as on its deep surface, i.e., the surface which will be juxtaposed to the recipient. These pigments are the same as those which are in current medical use for medical tattooing, such as in nipple-areola facsimile fabrication during surgical staged breast reconstruction. In this manner the site of application of the graft will always be identifiable, for the purpose of subsequent partial or complete graft removal after placement. In an alternative embodiment, the basal matrix of the graft is impregnated with non-toxic, insoluble, pharmaceutical-grade chromogen or fluorogen. The site of graft application is then identifiable, for ex ample, by surface illumination with light of the proper wavelength, e.g., Wood's lamp. Some members of the tetracycline family exhibit such properties, and would exemplify this embodiment.

1.6 Assessment of gene expression in modified cells. Once transfer of the nucleic acid of interest has been accomplished ex vivo under conditions such that the expressed protein is secreted extracellularly, the cells under culture conditions are assayed for production of translation product. Indirectly, i.e. by surrogate assessment, the genetic machinery is assumed to be functional if the cells have passed a stringent selection in the case of linking an antibiotic resistance marker gene into the DNA cassette. Further, if the cells express the linked marker genes, such as luciferase or β-galactosidase, they are again assumed to be functional in terms of producing the translation product for the therapeutic gene itself. It is also within the scope of the invention to directly measure extracellular secretion of the expressed protein. Appropriate direct assays of the therapeutic proteins are well known in the art, e.g., by ELISA, or immunolocalization. Tissue culture fluid samples can be assayed as a function of time to determine levels of production and secretion of the therapeutic protein. The results of the direct assays provide a means whereby the size, and the cell population density of the graft can be calculated in advance of graft preparation. This will allow tailoring of the therapeutic agent's dosage to the individual recipient's requirements. An exemplary assay would be a quantitative ELISA assay. The same assay would subsequently be conducted on blood samples taken from the recipient after graft application (in the case of a systemic circulating gene product) or on samples of cerebrospinal fluid (in the case of a dural or leptomeningeal graft), or alternatively on samples of body cavity fluid (in the case of body cavity graft application). In this way, the in vivo dosage delivered will be monitored. If the dosage is excessive, fractional removal of a portion of the graft can be performed as a minimally invasive external or endoscopic surgical procedure. Further, at the completion of the treatment, or if an adverse treatment reaction occurs, the entire graft can be removed in a similar fashion. The effect of the therapeutic protein or other compound can also be determined by those skilled in the art. Further, the invention is useful in a screen to identify a candidate agent that modulates the measurable effect of the secreted protein or compound. This screen can be performed in vivo, ex vivo or in vitro. The candidate agent may be an anti-inflammatory agent, an anti-neoplastic agent, an oncogene, a proto-oncogene, a cytokine, or an inflammatory mediator, which would include, e.g., any compound that increases or decreases inflammation.

1.7 Regulation of treatment. As part of the method of treatment, the amount and/or duration of the treatment can be regulated.

1.7.1 Amount of treatment. One method of regulating the amount of protein or compound produced by the cell preparation graft is by altering the size of the cell preparation graft introduced to the mammal, or by altering the number of cells contained within the cell preparation graft. Another method of regulating the amount of protein or compound produced by the cell preparation graft is to use expression vectors that vary in the levels of protein expressed, and/or in the number of functional gene copies which are incorporated in the modified cells.

1.7.2 Duration of treatment. In a preferred embodiment, the duration of the treatment can be controlled by removing the cell preparation graft introduced into the patient. This can be done surgically, chemically, or otherwise. In some embodiments, the cell preparation graft is detected with a detectable marker, e.g., GFP. In other preferred embodiments, the expression of the therapeutic proteins or compounds is regulated by a molecular switch, which can be any inducible event or process. This switch either increases or decreases the amount of the proteins or compounds produced by or secreted from the modified cells. Examples of molecular switches known in the art include the CRE/LOX system, the TET system, the NFkappaB/UV light system, the Leu3p/isopropylmalate system, and the GLVPc/GAL4 system (See e.g., Sauer, 1998, Methods 14(4): 381-92; Lewandoski, 2001, Nat. Rev. Genet 2(10):743-55; Legrand-Poels et al., 1998, J. Photochem. Photobiol. B. 45:1-8; Guo et al., 1996, FEBS Lett. 390(2):191-5; Wang et al., PNAS USA, 1999, 96(15):8483-8). The switch can also be an endogenous feedback loop, wherein the protein or compound produced either increases or decreases cellular production of itself. The switch can also be endogenous, such as by inhibition of transcription or translation by a drug or other compound. The switch also includes heat- and cold- or ultraviolet light-inducible expression systems. In a preferred embodiment, the expression of therapeutic proteins is modulated by exposure of the cell preparation graft to UV light. Promoter elements necessary for this regulation are known in the art (See. e.g., Hu et al., 2000, J. Biol. Chem. 275:2979-85).

2. Compositions.

In a Second aspect, the invention encompasses compositions of matter such as cell preparation grafts. These cell preparation grafts may comprise one or more cells. These cell preparation grafts may also include other components.

2.1 Extracellular matrix proteins and cell surface receptors. In some embodiments of the invention the cell preparation graft includes extracellular matrix proteins, e.g., collagens, laminins, fibronectin, vitronectin, tenascin, integrins, glycosaminoglycans, elastin and fibrin.

2.2 Growth factors and cytokines. In some embodiments of the invention the cell preparation graft includes growth factors and/or cytokines, such as vascular endothelial cell growth factors, platelet derived growth factors, epidermal growth factors, fibroblast growth factors, hepatocyte growth factors, insulin-like growth factors, and transforming growth factors. These factors may be contacted to the biocompatible matrix or the modified cells or both.

2.3 Matrix-degrading enzymes. In some embodiments of the invention the cell preparation graft includes matrix-degrading enzymes, e.g., matrix metalloproteinases, collagenases, keratinases, elastases, heparinases, heparanases, glucoronidases, heparitinases, hyaluronidases, sulfatases, dermatanases, chondroitinases, and other enzymes catalyzing the degradation of glycosaminoglycans and proteoglycans. These matrix-degrading enzymes may be contacted to the biocompatible matrix or the modified cells or both.

2.4 Bio-compatible polymers. The cell preparation graft of the invention includes a biocompatible matrix. This biocompatible matrix includes biocompatible polymers, such as natural and synthetic biopolymers. These polymers can be absorbable such as those constructed with a polyglactan mesh or with a collagen glycosaminoglycan matrix or they can be non-absorbable, such as Biobraneg, polypropylene (Marlex®), Dacron®, PTFE (GoreTex®), Medpor®, Proplast® or polyamide (Supramid®). Alternatively, cell preparation grafts can be suspended in fibrin glue and the fibrin-cell mixture can be contoured into a suitable format (e.g., a cylindrical tube, sac, sheet or fiber) and placed into a porous silicon polymer chamber which would allow egress of secreted gene products and influx of nutrients to the cells, while allowing facile implantation, containment of the transformed cells and subsequent removal.

2.5 Adhesive materials. In some embodiments of the invention the cell preparation graft includes adhesive materials such as peptides, glycopeptides, and natural and synthetic adhesive materials.

For a better understanding of the invention, reference is made to the below referenced examples following immediately hereafter.

### EXAMPLES

### Example 1

### Gene Therapy using Factor VIII Transformed Grafted Human Keratinocytes for Treatment of Hemophilia A

Human keratinocytes (hK) may be easily and safely obtained from the intended recipients of a particular gene therapy, and then subjected ex vivo to the appropriate genetic manipulation. Once modified, the hK may be returned to the intended recipient as an integral sheet graft of precisely defined dimensions, and precisely determined cell count of modified hK, all of which serve to precisely control the dosage of the particular therapy.

For example, under locally administered field block anesthesia using 2% plain lidocaine, and after standard anti-bacterial surgical skin preparation and draping, a thin split thickness skin graft biopsy (generally 0.010-0.014″ thickness) is harvested from a concealed, yet convenient donor site. The surface area of the graft is dictated by the cell number of hK required for genetic manipulation, keeping in mind that harvested basal hK can be serially sub-cultured to easily expand their numbers. Typically, a graft will be on the order of 2×2 cm in size, or even smaller. Such grafts include the entire epidermis, the epidermal-dermal basement membrane zone, and a very thin lamina of the subjacent papillary dermis. Harvesting of grafts encompassing the entire epidermis is preferred for optimal recovery of the more proliferative hK resident in the stratum basalis. This would be important if one is considering sub-culture expansion. Hemostasis at the skin graft donor site is secured by application of a sterile gauze sponge wetted with 1:100,000 epinephrine for 3-5 minutes. The donor site can be closed primarily, or alternatively treated as a normal skin graft donor site, dressing it with an adhesive semi-permeable membrane cover to facilitate healing of the donor site via natural re-epithelialization. The harvested skin shaving biopsy is maintained under sterile conditions, and transferred to a sterile petri dish containing 0.25% trypsin solution (Sigma, St. Louis, Mo.). The graft is incubated generally overnight in the trypsin solution at 4° C., at which temperature, tryptic activity is minimal, but the enzyme is allowed to permeate through the dermal side of the graft to the dermal-epidermal junction. The petri dish is then transferred to an incubator at 37° C. to accelerate enzymatic release of the epidermis from the dermis. The epidermal sheet is carefully separated from the subjacent dermis, avoiding contamination of the epidermis with any fibroblasts from the dermis. The epidermal sheet is then placed in a fresh petri dish also containing trypsin, where it is gently agitated and its undersurface scraped with a rubber policeman to remove the basal hK. The tryptic activity is then quenched by addition of any of a number of inhibitors, e.g., soybean trypsin inhibitor. The epidermal cells are then collected by centrifugation at low speed, and plated on plastic petri dishes coated with collagen, irradiated 3T3 cell layer, poly-lysine or any of a number of adhesion facilitating coatings. The primary hK are then grown to sub-confluence in any one of several commercially available keratinocyte nutrient media, generally at 37° C., in a 5% CO₂ atmosphere. The sub-confluent hK cell sheet may then be dissociated using EDTA solution or via enzymatic means (e.g., trypsin, dispase), and then serially expanded in sub-culture. In this manner, the original cell number of harvested hK can be geometrically expanded in a brief period of a few weeks. At any point, a portion of the expanded hK can be withdrawn and cryopreserved in a viable state in vapor phase liquid nitrogen using any of a number of cryo-protective agents (e.g., glycerol, DMSO), using techniques known in the art. The cryopreserved hK are then banked for later use in the same recipient, using either the same or even a different genetic manipulation.

To effect transformation of the hK, an expression vector containing a nucleic cassette which encodes a truncated version (B-domain deleted) of Factor VIII is prepared and introduced into an adeno-associated virus vector system as is commercially available (Strategene, La Jolla, Calif.), using techniques that are standard in the art. The Factor VIII can be obtained from plasmid pSAT.8ci. The plasmid is then inserted into the pAAV-hrGFP vector to allow for detection of expression in vivo using fluorescence-activated cell sorting (FACS) analysis or fluorescence microscopy.

Additional controlling elements are added to the cassette as may be required by needs of the intended recipient. These may include, for example, inducible promoters, and/or enhancers to thereby refine and control the levels of expression of the Factor VIII product. Likewise, Factor VIII secretion can be regulated by incorporation of a molecular switch, which can be any inducible event or process. These procedures are conducted using methodologies well known in the art. The vectors are purified and titered following methodologies known in the art.

The subconfluent hK are then transfected with the viral vector as constructed above, using exposure methods known in the art. The transformed hK are then sub-cultured in medium containing G418 (Geniticin; Gibco), at a concentration of 300 μG/mL to select the resistant colonies. Transformants are isolated and sub-cultured in standard nutrient medium. A pool of the transformants are subjected to standard multi-well Factor VIII assay in the culture medium to determine level of Factor VIII secretion. This assay provides the information which is required to calculate both the cell number to be incorporated into the clinical grafts, as well as allowing a first estimation of the dimensions of the graft to be applied.

For long term treatments when the transformed cells are applied externally to a suitably prepared recipient site (vide infra), a durable dermal matrix provides the best environment for the transformed hK. Deconstructed human dermis (DhD) is one ideal matrix (Demarchez, Transplantation, 1987). This is prepared by exhaustive, non-denaturing incubation of split-thickness skin grafts obtained from widely distributed clinical tissue banks, and rigorously screened by the banks, following FDA guidelines to minimize risk of disease transmission. The preparation of DhD effectively removes all cells and soluble allo-antigens. Alternatively, a commercially available devitalized allogeneic human dermis can be employed in a similar manner (Alloderm™ LifeCell). The DhD is subjected to sterile medical tattooing about its perimeter and deep surface, using commercially available surgical-grade pigments applied with surgical tattoo instrument (Permark, Permark Inc, Edison, N.J.). This allows unequivocal identification of the graft long after it has been applied to the intended recipient, for purposes of dose reduction through fractional graft excision, or termination of therapy via complete graft removal. Alternatively, in non-allergic recipients, the DhD is impregnated with an insoluble fluorochrome (such as an insoluble tetracycline analog) by which the graft can be identified under UV (Wood's) lamp.

It has been shown that DhD allows for rapid attachment of hK, as well as rapid re-establishment of the normal basement membrane zone. The transformed hK are counted with a hemocytometer. Quantitative assay of Factor VIII secretion per cell is performed by standard multi-well assay. The transformed hK are then plated onto the DhD which is maintained at the air-liquid interface in complete nutrient medium. The growth of the hK on the DhD is monitored by vital microscopy, and grafts are ready for clinical use once the hK have established confluency on the DhD. A second assay of Factor VIII secretion is conducted at this point to refine the Factor VIII dose calculation, and compute with accuracy the size of the graft to be applied to the intended recipient.

The prepared graft is now ready for clinical application. Under sterile operating conditions, the recipient area of the body is surgically prepared and draped, and anesthetized.

For most grafts, this can be accomplished via local field block anesthesia using 1-2% lidocaine. For larger grafts, appropriate nerve block anesthesia or Bier block anesthesia is employed. The recipient area is selected based upon a consideration of the size of the graft which is required, and optimal camouflage in a concealed, yet accessible body region. Most often, the upper thigh or buttocks region meets these criteria.

After suitable local or regional anesthesia is established, the intended recipient area is dermabraded using commercially available power instrument (Permark, Permark Inc. Edison, N.J.). The depth of the dermabrasion is to the level of the junction of the papillary dermis with the deeper reticular dermis. If the dermabrasion is more superficial, prompt re-epithelialization will occur, resulting in delamination of any applied over-graft. Only if most or all of the source of native, resident epidermal cells is removed will the applied graft endure long term. This means that not only does the epidermis itself need to be removed, but also the papillary dermis containing the adnexal structures which themselves are the source of epidermal cells for spontaneous re-epithelialization. This is well known in the art of skin grafting in the field of surgery.

Hemostasis at dermabraded intended recipient site is secured by application of sterile gauze sponges soaked in epinephrine solution, 1:100,000 dilution and/or commercially available topical thrombin spray (Jones Pharma, St. Louis, Mo.). The prepared graft is then applied as an integral sheet and secured about the perimeter with surgical skin staples or conventional absorbable surgical sutures. The graft is then dressed as would be any surgical skin graft, using a non-adherent petrolatum gauze (Xeroform, Sherwood, Davis&Geck, St. Louis, Mo.). Frictional trauma at the grafted area is avoided for at least 3-4 weeks after grafting to avoid shearing and disruption at the graft-host interface. Beyond that time, the graft care is no different than normal skin care and hygiene.

Factor VIII production is monitored via standard clinical serum assay. If levels are sub-optimal, additional segments of graft may be added. Alternatively, if Factor VIII production is excessive, a fraction of the graft may be removed via dermabrasion under local anesthesia. If the area is dermabraded to dermis, the area can be closed primarily.

### Example 2

### Introduction of Vascular Endothelial Cell Growth Factor (VEGF)

In this example, the goal is to provide therapeutic levels of VEGF to an extremity (upper or lower limb) of a patient experiencing ischemic symptoms (rest pain, claudication, ulceration) as the result of chronic peripheral vascular disease. The VEGF therapy can be performed as a stand-alone procedure in patients who are at unacceptable risk for any major surgical revascularization, or who are deemed “un-reconstructable” due to multi-level, diffuse obstructive pathology, or prior failed surgical reconstruction. Alternatively, the procedure can also be performed in conjunction with standard vascular reconstructive surgery.

The VEGF gene has been described (See, e.g., U.S. Pat. No. 5,332,671). VEGF stimulates angiogenesis in vivo, and effective therapeutic angiogenesis in both the coronary and peripheral vascular system of humans has been reported (Circulation, 1998;58:238 and Circulation, 2001;104:753)

The inclusion of a signal sequence preceding the amino terminus of VEGF allows for its secretion by intact cells. The strategy of this example is therefore to transform an easily obtained mesenchymal cell, the fibroblast, with the VEGF gene, followed by growth of the transformed cells in sufficient quantity to be able to provide the intended recipient with therapeutic levels of the gene product, and fabrication of a measurable graft for dose calculation and adjustment, and insertion of the graft in a minimally invasive independent procedure, or alternatively insertion during a vascular surgical operation.

Dermal fibroblasts are obtained from a small skin biopsy specimen taken from the intended VEGF recipient patient. The biopsy is performed in a concealed body region, remote from any ischemic zones. A preferred site is the axillary region, by reason of its accessibility and skin laxity which allows for excision of a full thickness specimen of skin followed by primary repair of the donor site. The axillary region is prepared and draped in sterile manner. Anesthesia is achieved via field block using 2% lidocaine. Next a small elliptically shaped full thickness skin biopsy is performed, orienting the long axis of the ellipse parallel with the normal so-called relaxed skin tension lines of the area in order to facilitate direct primary repair of the donor site. The size of the biopsy is generally on the order of 2×3 cm. Hemostasis of he donor site is secured using normal surgical techniques, and the donor site is then repaired in standard surgical layered technique, using absorbable sutures which do not require removal. The biopsy specimen is transported to the laboratory in a sterile container.

Dermal fibroblasts are recovered from the biopsy specimen via trypsin treatment. Generally, incubation in 0.25% trypsin at 4° C. overnight allows for optimal penetration of the enzyme into the entire dermis, with little actual tryptic activity. Next, the specimen is incubated for 15-30′ at 37° C. to initiate tryptic activity and release dermal cells. The cells are released from the specimen by intermittent agitation of the specimen during the incubation. Tryptic activity is then terminated by the addition of soybean trypsin inhibitor. A cell pellet is then collected by low speed centrifugation of the cell solution. The cells are suspended in DMEM containing 10% serum or serum equivalent, counted in a hemocytometer and then plated in standard plastic flasks or petri dishes at an initial seeding level of 50-75×10³ cells/cm². Cell are grown at 37° C. in 5% CO₂ atmosphere. When the seeded surfaces reach 70-80% confluence, cells are released with brief trypsinization, and serially sub-cultured under the same conditions.

Once the number of 1^{st} passage fibroblasts has reached a suitable level for therapy of the intended recipient, the transformation step is taken. The choice of methodology is dictated by the needs of the intended recipient. For example, in a patient with end-stage, non surgically reconstructable peripheral vascular disease, associated with rest symptoms and skin ulcerations, a preferred strategy may be to integrate the VEGF gene into the recipient genome using a retroviral vector to thereby enhance the duration of sustained expression and delivery of gene product. On the other hand, in a patient undergoing vascular reconstruction, an episomal delivery of the gene-containing nucleic acid cassette may be preferred for shorter term delivery of therapy. In this example, short term therapy is illustrated.

The transfection of a packaging cell line, collection of high-titer recombinant virus from the cell lysates, and transduction of target cells (dermal fibroblasts) with the VEGF gene may be accomplished by any of a number of approaches, all within the scope of individuals with skill in the art. The AAV Helper-Free System (Stratagene, La Jolla, Calif., and U.S. Pat. Nos. 5,622,856, 5,945,335, 6,001,650, 6,004,797 and 6,027,931) is but one preferred strategy. Alternatively, full length cDNA for human VEGF can be directionally cloned into the EcoRI site of pcDNA3.1 vector (Invitrogen Corporation). Hemagglutinating virus of Japan (HVJ) cationic liposome can be prepared by mixing 200 μg DNA with a lipid film of 10 mg of a mixture of phosphatidylserine, dimethylaminoethane-carbamoyl cholesterol and cholesterol to generate a liposome-DNA suspension. The suspension is then treated with 30,000 hemagglutinating units of inactivated HVJ by ultraviolet light resulting in 1 ml of HVJ-liposome (See Circulation 2001;104: I207). Dermal fibroblast cultures at 50% confluence are plated in a 165 cm flask are incubated with 450 ml of the HVJ liposome containing the human VEGF gene in pcDNA3.1 in 20 ml of proliferation media for two hours. The transformants are isolated and subcultured

The replication-deficient AAV vector, pAAV-LacZ, is cut with NotI, the LacZ cassette is lost and the prepared expression cassette, harboring the VEGF and neomycin resistance genes, from the pCMV-MCS vector is cloned into the complementing NotI sites of this vector. The recombinant vector is then co-transfected into a viral packaging cell line E1a-partial Eb-adenovirus to produce recombinant, replication-deficient AAV virions. The early passage dermal fibroblasts are then transfected under standard conditions for AAV transfection, as known to those skilled in the art. The transformants are selected by sub-culture in medium containing G418 (Geniticin; Gibco), at a concentration of 300 μG/mL to select the resistant colonies. Transformants are isolated and sub-cultured in standard nutrient medium. A pool of the transformants are subjected to standard multi-well VEGF ELISA assay (Chemicon International, Temecula, Calif.) in the culture medium to determine level of VEGF secretion. This assay provides the information which is required to calculate both the cell number to be incorporated into the clinical grafts, as well as allowing a first estimation of the size of the graft to be applied.

Selection of the graft matrix to carry the transformed cells in known numbers, and dose of gene expression is based upon the characteristics of the transformed cells and the tissue bed into which the graft will be placed. In this example, Biobrane®, a non-absorbable nylon matrix capable will be employed to facilitate rapid in-growth of the transformed fibroblasts in high numbers, i.e., high yield of transplantable functional gene copies. Biobrane® has been used as a matrix for neonatal fibroblasts in the construction of dermal facsimiles (Surgery 1994; 115:633).

The nylon mesh is prepared for seeding with the transformed fibroblasts by sterilization, and soaking in fibroblast culture medium (DMEM plus 10% calf serum). Once the transformed cells have been grown in sufficient quantity, as determined by multi-well VEGF assay again using a VEGF ELISA assay kit (Chemicon International) to determine approximate quantitative VEGF secretion per cell, sub-confluent cultures are mechanically dissociated, pelleted and re-suspended in DMEM, and counted. The mesh is then seeded via injection of a known quantity of transformed cells into its core. Clinical experience with VEGF to date indicates that a therapeutic effect can be achieved with incorporation of between 4×10⁵ and 4×10¹⁰ Advegf units into a graft, or on the order of 2000 μg of VEGF vector (See Circulation 1998;97:1114). Cells are then allowed to adhere to matrix surfaces by culture at 37° C. in 5% CO₂ in complete medium for an overnight period.

When the graft is ready for the intended recipient, the grafting procedure is performed. When done as an independent procedure, i.e., no surgical revascularization, the graft may be placed under extremity nerve block anesthesia with sedation, spinal/epidural anesthesia or general anesthesia, depending upon patient circumstances. Under appropriate anesthesia, the fascia lata area of the affected lower extremity is surgically prepared and draped. A small incision is made and careful blunt, spreading dissection is carried down to the external surface of the fascia lata. Tunneling dissection is then performed distally to expose a vertical strip of the fascia lata. This is facilitated through the us of a dissecting endoscope (Stryker, Kalamazoo, Mich.), much as is done for regional cutaneous flap elevations for aesthetic surgery procedures. A longitudinally oriented fasciotomy of the fascia lata is performed with a small curved scalpel blade, exposing the surface of the subjacent tensor fascia lata muscle. No ill consequence is sustained as a result of the fasciotomy, as evidenced by the many thousands of fascia lata strip grafts which have been harvested in similar fashion for use in reconstructive surgery procedures for nearly a hundred years.

The exposed surface of the tensor fascia lata muscle is an ideal bed for the VEGF therapy delivery as reports have shown (See Circulation, 2001:104:753). The graft of appropriate length is inserted longitudinally, with the endoscopic assist or with the aid of a narrow lighted retractor to elevate the overlying skin from the exposed TFL muscle, thereby allowing accurate placement of the graft. If more than one graft is required, the additional grafts are placed adjacent to one another, all lying on the muscle surface. Once the surgical retractor and/or endoscope are withdrawn, the grafts are held securely in place by the natural tension of the overlying skin. The small surgical incision is repaired in layers using absorbable sutures.

The recipient is followed serially for signs of clinical improvement, via decreased rest pain or claudication, improvement in healing of any ulcers, serial non-invasive vascular studies, including trans-cutaneous pCO₂, and ankle brachial indices. as well as serial VEGF determinations in serum (R&D Systems, Minneapolis, Minn.).

If an initial clinical improvement is followed by a regression which is attributed to waning VEGF levels produced by the grafts, a secondary procedure can easily be performed, with removal of the spent grafts and insertion of freshly prepared ones. This is best executed with some pre-planning, and can be done relatively quickly if a portion of the stock of the transformed fibroblasts is withheld and cryopreserved at the time of the first procedure. The fibroblasts can be preserved in DMEM plus 25% calf serum and (10% concentration of glycerol or DMSO), and then maintained in vapor phase liquid nitrogen until needed. At that time, vials of cryopreserved transformed fibroblasts are thawed, and plated in DMEM plus 10% calf serum, and grown in culture at 37° C. in 5% CO₂. As described above, when sufficient cell numbers are available, new grafts are prepared, and surgically implanted.

If, for any reason, a recipient patient experiences an untoward reaction to the grafts, these can be surgically removed in their entirety, by simply re-establishing the original surgical incision in the thigh, exposing the grafts and removing them.

This strategy is also useful in other body regions to enhance vascularity in a setting of ischemia. The outer surface of cardiac muscle is exposed to the inner lining of the pericardial sac. The sac is routinely surgically opened during the conduct of coronary artery bypass procedures. This exposure is an ideal means whereby VEGF cardiac therapy can be implemented as an adjunct to the bypass operation itself. Alternatively, the patch could be applied to the pericardium using minimally invasive thoracoscopic or mediastinoscopic techniques.

For planned, elective bypass surgery, dermal fibroblasts are harvested, serially expanded, and transformed, as described above. The transformed fibroblasts are then integrated within the matrix of DhD, or other natural, low biodegradable structural protein matrix (for example, human fascia lata obtained from tissue bank sources, and deconstructed as is dermis, or alternatively a laboratory prepared collagen sheet). At the time of coronary bypass surgery, the integrated graft is sewn into a window prepared in the pericardial sac. The reticular dermal side of the DhD is oriented to face the myocardial muscle, as this is the more porous surface which will facilitate diffusion of VEGF from the modified fibroblasts within. Since the pericardium is not readily accessible again after cardiac surgery, this construct would preferably contain in the insertion cassette, one or more biological switch genes to provide for regulation or cessation of VEGF therapy.

### Example 3

### CNS Delivery of CNTF

The blood brain barrier is a biologic shield which protects the brain from foreign substances and maintains a constant, tightly regulated environment for the brain. The barrier provides a significant challenge to the treatment of neurological disorders—both oncological and degenerative—through systemic drug delivery or administration. This example describes a grafting technique, using transformed autologous fibroblasts to provide human ciliary neurotropic factor (CNTF) for the treatment of neurodegenerative disorders.

CNTF is potent neural factor initially originally characterized as a survivability factor for chick ciliary neurons in vitro. More recently, CNTF has been shown to promote survivability and differentiation of other neuronal cell types. Systemic administration of CNTF has proven unsuccessful for the treatment of neurological disorders. However, intrathecal administration of CNTF has been found to provide significant protective effects against neurodegenerative diseases including Huntington's disease (Mittoux, Human Gene Therapy 2000;11:1177) and amyotrophic lateral sclerosis (Aebischer, Nature Medicine 1996;2:696). CNTF also has appetite suppressive properties and has been used as in the treatment of obesity in laboratory animals. In addition, the presence of the herpes simplex thymidine kinase gene (HSV-tk) in the statement vector provides a way to eliminate these fibroblasts which upon exposure to gancyclovir, therefore providing a mechanism of exogenous control of gene statement.

Under local anesthesia (2% lidocaine) and sterile skin preparation a full thickness (including epidermis and dermis) sample of human skin can be harvested as a source for fibroblasts. The donor site can be any location in the body where there would be an inconspicuous scar such as the groin crease or axilla. The donor defect can be closed primarily using an absorbable monofilament suture. The skin sample is then transported sterile to the laboratory for fibroblast isolation and culture.

Dermal fibroblasts are recovered from the biopsy specimen via trypsin treatment. Generally, incubation in 0.25% trypsin at 4° C. overnight allows for optimal penetration of the enzyme into the entire dermis, with little actual tryptic activity. Next, the specimen is incubated for 15-30 min at 37° C. to initiate tryptic activity and release dermal cells. The cells are released from the specimen by intermittent agitation of the specimen during the incubation. Tryptic activity is then terminated by the addition of soybean trypsin inhibitor. A cell pellet is then collected by low speed centrifugation of the cell solution. The cells are suspended in DMEM containing 10% serum or serum equivalent, counted in a hemocytometer and then plated in standard plastic flasks or petri dishes at an initial seeding level of 50-75×10³ cells/cm². Cells are grown at 37° C. in 5% CO₂ atmosphere. When the seeded surfaces reach 70-80% confluence, cells are released with brief trypsinization, and serially sub-cultured under the same conditions. The fibroblasts are then transfected. The hCNTF gene is cloned into an statement vector containing a mutant dihydrofolate reductase gene (PNUT) and modified as described by Deglon (Human Gene Therapy 1996;7:2135). The transcription of the hCNTF gene is driven by the metallothionein-1 promoter and uses the human growth hormone polyadenylation signal sequences for 3′ processing. The 150-p XbaI-EcoRI fragment of the immunoglobulin signal peptide is fused to the EcoRI site at the position of the natural hCNTF initiation codon to ensure secretion of the factor. The plasmid is then modified by introducing the herpes simplex virus thymidine kinase gene (HSV-tk) at the unique Not I site. The vector is further modified by inserting the neomycin resistance gene under the control of an SV40 promoter at the BamHI-Not 1 site. Transfection is then accomplished by electroporation.

Fibroblasts are trypsinized and rinsed from the cell culture flask using culture medium. The cells are centrifuged and the supernatant is removed. The cell pellet is then resuspended in electroporation buffer (20 mM HEPES pH 7.3, 137 mM NaCl, 5 mM KCl, 0.7 mM Na₂HPO₄, 6 mM dextrose). The cells are re-centrifuged, the supernatant discarded and the cells resuspended in the electroporation buffer supplemented with bovine serum albumin (1 mg/ml). The plasmid is then added to the electroporation curette and cells are added so that there is a DNA concentration of 120 μg/0.5 ml of cell suspension. Optimal electroporation conditions are determined with minimal experimentation, as the technique is well known in the art. Using readily available commercial apparatus (e.g., GenePulser, BioRad, Hercules,Calif.), a representative set of experimental parameters would be a voltage of 200-300V, and a capacitance of 750-950 μF. Pulsing time is varied to maximize stable DNA incorporation, and minimize lethal voltage effects. Starting pulse times are on the order of 7-15 mS. Transfected cells are then selected with G-418 (Geneticin) as described in Example 1 above.

The transformed cells are then grown to subconfluence and lightly trypsinized to effect their separation. After trypsin neutralization, the cells are counted and then pelleted at low speed centrifugation. The cells are then resuspended in a small volume of DMEM+10% serum and then concentrated cell suspension is mechanically spread onto the reticular surface of the acellular dermal matrix known as DhD as described in Example 1. The transformed fibroblasts are then nurtured in their dermal matrix environment at 37° C. in 5% CO₂. Punch biopsies of known dimensions are cultured in miniwells for 24 hr, and quantitative CNTF production and secretion into the medium is determined via ELISA assay using the CNTF ELISA kit (R & D Systems, Minneapolis, Minn.). In this manner, the dose, i.e., size, of the graft can be computed based upon the need of the intended recipient.

Once a DhD graft of appropriate dimensions has been constructed, surgical implantation is undertaken. The planning of the implantation is undertaken by the neurology and neurosurgery medical staff. Once, a site has been selected, a burr hole craniotomy is performed, a procedure which can often be done under local anesthesia with conscious sedation. An incision is made in the dura. The DhD patch is then sewn into the incisional defect created. The patch is secured in place using non-absorbable sutures which are dyed so as to allow subsequent identification of the patch graft at a later date if necessary. Production of CNTF is quantified using ELISA assay of cerebrospinal fluid obtained from lumbar puncture, performed serially, and compared with baseline CNTF levels in CSF determined pre-operatively. The production of CNTF can be regulated by removal of a portion or the entire patch. Alternatively, CNTF production can be regulated by the administration of oral gancyclovir because of the inclusion of the HSV-tk sequence in the CNTF plasmid. Drug administration and dosage are determined in conjunction with neurology staff, and based upon clinical response to the CNTF therapy and CNTF levels in CSF.

### Other Embodiments

Having thus presented the present invention in view of the above described embodiments, various alterations, modifications and improvements will readily occur to those skilled in the art. Such alterations, modifications and improvements are intended to be within the scope and spirit of the invention. Accordingly, the foregoing description is by way of example only and is not intended to be limiting. The invention's limit is defined only in the following claims and the equivalents thereto.

We claim:

1. A method of reversibly delivering a cell preparation graft to a mammal, the method comprising:
introducing a cell preparation graft comprising one or more cells and a biocompatible matrix into a tissue of a mammal; and
removing said cell preparation graft or a portion thereof from said tissue.

2. The method of claim 1, wherein said one or more cells are modified cells.

3. The method of claim 1, wherein said tissue is skin.

4. The method of claim 2 wherein said one or more modified cells are selected from the group consisting of keratinocytes, keratinocyte precursor cells, keratinocyte stem cells, epithelial cells, fibroblasts, endothelial cells, pericytes, periosteal cells, osteocytes, osteoblasts, perichondrium, chondrocytes, chondroblasts, serosal cells, hepatocytes, pancreatic islet cells, amniotic membrane cells, myocytes, myoblasts, skeletal joint synovium cells, pericardial cells, dural cells, meninges cells, glial cells, neural cells, amniotic and placental membrane cells, fetal and adult derived stem cells.

5. The method of claim 2, wherein said one or more modified cells are keratinocytes.

6. The method of claim 2, wherein said modified cells are selected from the group consisting of autologous cells, embryonic cells, neonatal cells, allogeneic and/or xenogeneic cells which are genetically or otherwise modified as may be required to induce tolerance in the intended recipient.

7. The method of claim 2, wherein said modified cells express a recombinant protein.

8. The method of claim 7, wherein said recombinant protein is selected from the group consisting of insulin, insulotropin, glucagon, human growth hormone, erythropoietin, hemoglobin, leptin, adenosine deaminase, purine nucleoside phosphorylase, CD-4, Factor VIII, Factor IX, α₁-antitrypsin, LDL receptor protein, glucose-6-phosphatase, albumin, B-glucosidase, cystic fibrosis transmembrane conductance regulator, tissue plasminogen activator, urokinase, streptokinase, antithrombin, apolipoprotein, low density lipoprotein receptor, vascular endothelial growth factor, calcitonin, parathyroid hormone, parathyroid hormone-like hormone, adenosine deaminase, phenylalanine hydroxylase, von Willebrand Factor, interleukin, interferon, p53, anti-BRCA, anti-VEGF, anti-epidermal growth factor, endothelin, ciliary neurotropic factor, brain-derived neurite factor, neurite growth factor, tyrosine hydroxylase, lactase, transforming growth factor, granulocyte-colony stimulating factor, fibroblast growth factor, insulin-like growth factor, hirudin, thryoglobulin, thyroid hormone, dystrophin, lipid binding protein, L-asparaginase, pepsin, trypsin, chymotrypsin, cholecystokinin, sucrase, carboxypeptidase, catalase, uricase, elastase, thrombopoietin, adenosine deaminase, porphobilinogen deaminase, glutaryl CoA dehydrogenase, copper transporting ATPase, cystathionine B-synthase, globin, sonic hedgehog protein, protein antigens and immunoglobulins.

9. The method of claim 2, wherein the modified cells express a plurality of recombinant proteins.

10. The method of claim 9, wherein said plurality of recombinant proteins are expressed from a single vector.

11. The method of claim 9, wherein said plurality of recombinant proteins are expressed from a plurality of vectors.

12. The method of claim 2, wherein said modified cells synthesize and secrete a compound which is not a protein.

13. The method of claim 12, wherein said compound is selected from the group consisting of hormones, steroids, neurotransmitters, and natural products.

14. The method of claim 12, wherein said compound is an anti-neoplastic agent.

15. The method of claim 12 wherein said compound is selected from the group consisting of estrogens, androgens, anabolic steroids, digitalis glycosides, vincristine, doxorubicin, tamoxifen, and methotrexate.

16. The method of claim 7, where the amount of the recombinant protein expressed is regulated by the size of the cell preparation graft introduced.

17. The method of claim 7, where the amount of the recombinant protein expressed is regulated by the number of cells in the cell preparation graft introduced.

18. The method of claim 7, where the amount of the recombinant protein expressed is regulated by the number of copies of a nucleic acid incorporated into each cell of the cell preparation graft introduced.

19. A method of reversibly delivering a cell preparation graft to a mammal, the method comprising:
introducing a cell preparation graft comprising keratinocytes and a biocompatible matrix into the skin of a mammal; and
removing said cell preparation graft or a portion thereof from said skin.

20. A method of reversibly delivering a cell preparation graft to a mammal, the method comprising:
isolating one or more cells from said mammal;
modifying said cells;
integrating said modified cells with a biocompatible matrix to form a cell preparation graft;
introducing said cell preparation graft into said mammal; and
removing said cell preparation graft or a portion thereof from said mammal.

21. A method of modulating the delivery of a compound to a mammal, the method comprising:
introducing a cell preparation graft comprising one or more modified cells and a biocompatible matrix into a tissue of a mammal, wherein said modified cells are capable of secreting a compound into said tissue; and
removing said cell preparation graft or a portion thereof from said tissue so that the secretion of the compound is reduced or eliminated,
thereby modulating the secretion of said compound into said tissue.

22. The method of claim 21, wherein the amount of compound delivered is regulated by the size of the cell preparation graft introduced.

23. The method of claim 21, where the amount of the compound delivered is regulated by the number of cells in the cell preparation graft introduced.

24. The method of claim 21, where the amount of the compound delivered is regulated by the number of copies of a nucleic acid incorporated into each cell of the cell preparation graft introduced.

25. The method of claim 21, wherein the compound is selected from the group consisting of insulin, insulotropin, glucagon, human growth hormone, erythropoietin, hemoglobin, leptin, adenosine deaminase, purine nucleoside phosphorylase, CD-4, Factor VIII, Factor IX, α₁-anti trypsin, LDL receptor protein, glucose-6-phosphatase, albumin, B-glucosidase, cystic fibrosis transmembrane conductance regulator, tissue plasminogen activator, urokinase, streptokinase, antithrombin, apolipoprotein, low density lipoprotein receptor, vascular endothelial growth factor, calcitonin, parathyroid hormone, parathyroid hormone-like hormone, adenosine deaminase, phenylalanine hydroxylase, von Willebrand Factor, interleukin, interferon, p53, anti-BRCA, anti-VEGF, anti-epidermal growth factor, endothelin, ciliary neurotropic factor, brain-derived neurite factor, neurite growth factor, tyrosine hydroxylase, lactase, transforming growth factor, granulocyte-colony stimulating factor, fibroblast growth factor, insulin-like growth factor, hirudin, thryoglobulin, thyroid hormone, dystrophin, lipid binding protein, L-asparaginase, pepsin, trypsin, chymotrypsin, cholecystokinin, curase, carboxypeptidase, catalase, uricase, elastase, thrombopoietin, adenosine deaminase, porphobilinogen deaminase, glutaryl CoA dehydrogenase, copper transporting ATPase, cystathionine B-synthase, globin, sonic hedgehog protein, protein antigens, immunoglobulins, and natural products.

26. A method of treatment of a subject suffering from or at risk of a disease, the method comprising:
introducing a cell preparation graft comprising one or more modified cells into a tissue of the subject, wherein said modified cells secrete a therapeutic compound into said tissue; and
removing said cell preparation graft or a portion thereof from said tissue

27. The method of claim 26, wherein the amount of a therapeutic compound delivered is regulated by the size of the cell preparation graft introduced.

28. The method of claim 26, where the amount of the compound delivered is regulated by the number of cells in the cell preparation graft introduced.

29. The method of claim 26, where the amount of the compound delivered is regulated by the number of copies of a nucleic acid incorporated into each cell of the cell preparation graft introduced.

30. The method of claim 26, wherein the therapeutic compound is a recombinant protein.

31. The method of claim 26, wherein the disease or physiologic state is selected from the group consisting of diabetes, altered glycemic states, skeletal growth retardation, anemia, obesity, immunodeficiency, hemophilia, emphysema, hypercholesterolemia, pernicious anemia, glycogen storage disease, hypoalbuminemia, Gaucher's disease, cystic fibrosis, cardiovascular disease, calcium mineral diseases, Severe Combined Immunodeficiency, phenylketonuria, von Willebrand's disease, cancer, peripheral vascular disease, neurodegenerative disease, post-neural trauma, retarded fracture healing, lactose insufficiency, abnormal wound healing, keloids, thrombosis, hypercoagulability, diabetes insipidus, psychiatric disorders, pain, endocrineopathy, hypothyroidism, muscular dystrophy, naive immune state to pathogens, infection, shock, sepsis, leukemia, digestive disorders, pancreatic disorders, autoimmune disorders, oxidative stress, neurodegenerative disorders, hypouricasemia; gout, Ehlers Danlos disease, thrombocytopenia, SCID/ADA deficiency, porphyria, inborn errors of carboxylic and amino acid metabolism, glutaric acidemia, homocystinuria, Wilson's Disease, Menke's Disease, thalassemia, sickle cell anemia, and baldness.

32. A method of modulating the delivery of a compound to a mammal, the method comprising:
introducing a cell preparation graft comprising one or more modified cells and a biocompatible matrix into a tissue of a mammal, wherein said modified cells are capable of secreting a compound into said tissue; and
introducing an agent to said mammal, such that the secretion of the compound is reduced or eliminated without removal of all or a portion of the cell preparation graft,
thereby modulating the secretion of said compound into said tissue.

33. The method of claim 32, wherein said agent is introduced systemically.

34. The method of claim 32, wherein said agent is introduced locally at or near the introduced cell preparation graft.

35. The method of claim 32, wherein said agent acts by modulating gene expression in the modified cells.

36. The method of claim 32, wherein said agent is cytotoxic to said modified cells.

37. The method of claim 32, wherein said agent is selected from the group consisting of UV light, isopropylmalate, and GAL4.

38. A method of modulating the delivery of a compound to a mammal, the method comprising:
introducing a cell preparation graft comprising one or more modified cells and a biocompatible matrix into a tissue of a mammal, wherein said modified cells are capable of secreting a compound into said tissue; and
exposing said mammal to ultraviolet light, such that the secretion of the compound is modified without removal of all or a portion of the cell preparation graft,
thereby modulating the secretion of said compound into said tissue.

39. The method of claim 1, wherein the cell preparation graft comprises two or more subsets of modified cells, said subsets comprising one or more cells and functioning independently of other subsets.

40. The method of claim 39, wherein said cells of said subsets are the same cell type.

41. The method of claim 39, wherein said cells of said subsets are different cell types.

42. The method of claim 39, wherein the cell preparation graft is used to produce a plurality of compounds.

43. The method of claim 42, wherein the plurality of compounds act as a vaccine.

44. A method of identifying a candidate agent in a mammal, the method comprising:
introducing a cell preparation graft comprising one or more modified cells and a biocompatible matrix into a tissue of a mammal, wherein said modified cells are capable of secreting a compound into said tissue, said compound having a measurable effect on said tissue; and
introducing a candidate agent to said mammal, such that said effect of said compound is modulated without removal of all or a portion of the cell preparation graft,
thereby identifying a candidate agent.

45. The method of claim 44, wherein said modified cells secrete a plurality of compounds into said tissue.

46. The method of claim 45, wherein said plurality of compounds have a plurality of measurable effects on said tissue.

47. The method of claim 44, wherein the candidate agent is selected from the group consisting of anti-inflammatory agents, anti-neoplastic agents, oncogenes, proto-oncogenes, cytokines, and inflammatory mediators.

48. The agent identified by the method of claim 44.

49. A method of identifying a candidate agent, the method comprising:
contacting a candidate agent and a cell preparation graft, wherein said graft comprises one or more modified cells and a biocompatible matrix, wherein said modified cells are capable of secreting a compound, and wherein said compound is capable of having a measurable effect on a cell or a tissue of a mammal,
such that the secretion of said compound is modulated, thereby identifying a candidate agent.

50. The method of claim 49, wherein said contacting is performed in vitro.

51. The method of claim 49, wherein the candidate agent is selected from the group consisting of anti-inflammatory agents, anti-neoplastic agents, oncogenes, proto-oncogenes, cytokines, and inflammatory mediators.

52. The agent identified by the method of claim 49.

53. A cell preparation graft for introduction into a tissue of a mammal, said preparation graft comprising:
one or more modified cells capable of expressing a recombinant protein, hormone or a natural product;
a biocompatible matrix; and
a moiety capable of being detected in vivo.

54. The cell preparation graft of claim 53, wherein said moiety is a light-generating protein.
